(19) United States
(12) Patent Application Publication
(10) Pub. No.: US 2007/0149631 A1
(43) Pub. Date: Jun. 28, 2007

(54) Base-catalyzed alkoxylation in the presense of polyoxyethylene-containing compounds

(76) Inventors: Karl W. Haider, Wexford, PA (US); Jose F. Pazos, Charleston, WV (US)

Correspondence Address: BAYER MATERIAL SCIENCE LLC, 100 BAYER ROAD, PITTSBURGH, PA, 15205, US

(21) Appl. No.: 11/315,517

(22) Filed: Dec. 22, 2005

(51) Int. Cl. C08G 18/00; C08F 20/00; C07C 43/11

(52) U.S. Cl. 521/172; 568/679; 525/438

(57) ABSTRACT

The present invention provides a long-chain polyether polyol having a number average molecular weight of more than about 1,200 g/mole and produced by alkoxylating an initiator with an alkylene oxide in the presence of a basic catalyst having at least one cation thereof chelated with about 0.5 to about 20 wt. % of a polyoxyethylene-containing compound having a molecular weight of less than about 10,000 g/mole, wherein the weight percentage is based on the weight of the long-chain polyether polyol. The long-chain polyether polyols of the present invention may find use in providing flexible polyurethane foams and non-cellular polyurethanes.

### FIELD OF THE INVENTION

The present invention relates in general to polyether polyols, and more specifically, to a long-chain polyether polyol having a number average molecular weight of more than about 1,200 g/mole and produced by alkoxylating an initiator in the presence of a basic catalyst having at least one cation thereof chelated with from about 0.5 wt. % to about 20 wt. % of a polyoxyethylene-containing compound having a molecular weight of less than about 10,000 g/mole.

### BACKGROUND OF THE INVENTION

It has been known for many years that cyclic ethers complex potassium ions strongly. Crown ethers were discovered in the 1960's by Charles Pederson and he was awarded the Nobel Prize in 1987 for his efforts. The ability of cyclic ethers to strongly complex metal ions led to much scientific work. Unfortunately, because of the synthetic difficulty, high cost and high toxicity of these compounds, crown ethers have never found wide commercial application. Perhaps, because crown ethers were discovered first, most of those skilled in the art have overlooked the strong complexing abilities possessed by non-cyclic polyethers. Among the other advantages of non-cyclic polyethers are ready availability, low cost and the fact that polymers and oligomers of ethylene oxide are so non-toxic as to be acceptable food additives.

A commonly-assigned U.S. patent application also filed on an even date herewith and entitled “Base-catalyzed alkoxylation in the presence of non-linear polyoxyethylene-containing compounds”, (Atty. Docket No. PO8709, U.S. Ser. No. ______) discloses a non-linear, at least trifunctional polyoxyethylene-containing additive as a chelating agent for the base-catalyzed alkoxylation of long-chain polyethers, with no detrimental effect on flexible foams produced therefrom.

A second commonly-assigned U.S. patent application also filed on an even date herewith and entitled “Short chain polyether polyols for rigid polyurethane foam”, (Atty. Docket No. PO8707, U.S. Ser. No. ______) discloses a polyoxyethylene-containing additive as a chelating agent in the alkoxylation of short chain polyethers.

Finally, a third commonly-assigned U.S. patent application also filed on an even date herewith and entitled “Long-chain polyether polyols”, (Atty. Docket No. PO8706, U.S. Ser. No. ______) discloses a polyoxyethylene-containing initiator as a chelating agent in the alkoxylation of long-chain polyethers.

Although the concept of using linear polyethylene glycols (“PEGs”) for rate enhancement of the KOH catalyzed alkoxylation of long-chain polyols is known in the art (See “Synthesis of Polyether Polyols for Flexible Polyurethane Foams with Complexed Counter-Ion” by Mihail Ionescu, Viorica Zugravu, Ioana Mihalache and Ion Vasile, Cellular Polymers IV, International Conference, 4th, Shrewsbury, UK, Jun. 5-6, 1997. Paper 8, 1-8. Editor(s): Buist, J. M.), there are no published reports describing the influence of PEG molecular weight on its ability to accelerate the base-catalyzed alkoxylation of long-chain polyether polyols nor on the quality of the resulting long-chain polyether polyol.

It would therefore be desirable to provide teaching on the molecular weight dependence of the effectiveness of polyoxyethylene containing additives as reaction accelerators for producing long-chain polyether polyols by base catalyzed alkoxylation and to describe the effect of the molecular weight of those additives on the product quality of the resulting long-chain polyether polyols.

### SUMMARY OF THE INVENTION

Accordingly, the present invention provides a long-chain polyether polyol having a number average molecular weight of more than about 1,200 g/mole and produced by alkoxylating an initiator with an alkylene oxide in the presence of a basic catalyst having at least one cation thereof chelated with from about 0.5 to about 20 wt. % of a polyoxyethylene-containing compound having a molecular weight of less than about 10,000 g/mole, wherein the weight percentage is based on the weight of the long-chain polyether polyol. The inventive polyols may be used to provide flexible polyurethane foams and non-cellular polyurethanes.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, OH numbers, functionalities and so forth in the specification are to be understood as being modified in all instances by the term “about.” Equivalent weights and molecular weights given herein are number average equivalent weights and number average molecular weights respectively, unless indicated otherwise.

The present invention provides a long-chain polyether polyol having a number average molecular weight of more than 1,200 g/mole and produced by alkoxylating an initiator with an alkylene oxide in the presence of a basic catalyst having at least one cation thereof chelated with from 0.5 to 20 wt. % of a polyoxyethylene-containing compound having a molecular weight of less than 10,000 g/mole, wherein the weight percentage is based on the weight of the long-chain polyether polyol.

The present invention further provides a process for producing a long chain polyether polyol having a number average molecular weight of more than 1,200 g/mole and involving alkoxylating an initiator with an alkylene oxide in the presence of a basic catalyst having at least one cation thereof chelated with from 0.5 to 20 wt. % of a polyoxyethylene-containing compound having a molecular weight of less than 10,000 g/mole, wherein the weight percentage is based on the weight of the long-chain polyether polyol.

The present invention still further provides a flexible polyurethane foam made from the reaction product of at least one polyisocyanate and at least one long-chain polyether polyol having a number average molecular weight of more than 1,200 g/mole and produced by alkoxylating an initiator with an alkylene oxide in the presence of a basic catalyst having at least one cation thereof chelated with from 0.5 to 20 wt. % of a polyoxyethylene-containing compound having a molecular weight of less than 10,000 g/mole, optionally in the presence of at least one of blowing agents, surfactants, cross-linking agents, extending agents, pigments, flame retardants, catalysts and fillers, wherein the weight percentage is based on the weight of the long-chain polyether polyol.

The present invention also provides a process for producing a flexible polyurethane foam involving reacting at least one polyisocyanate and at least one long-chain polyether polyol having a number average molecular weight of more than 1,200 g/mole and produced by alkoxylating an initiator with an alkylene oxide in the presence of a basic catalyst having at least one cation thereof chelated with from 0.5 to 20 wt. % of a polyoxyethylene-containing compound having a molecular weight of less than 10,000 g/mole, optionally in the presence of at least one of blowing agents, surfactants, cross-linking agents, extending agents, pigments, flame retardants, catalysts and fillers, wherein the weight percentage is based on the weight of the long-chain polyether polyol.

By “long-chain” polyether polyol, the inventors herein mean a polyether polyol having a number average molecular weight of greater than 1,200 g/mole, preferably from 1,200 to 50,000 g/mole, more preferably from 1,200 to 30,000 g/mole, and most preferably from 1,200 to 8,000 g/mole. The molecular weight of the inventive polyols may be in an amount ranging between any combination of these values, inclusive of the recited values.

The long chain polyether polyols of the present invention are made by basic catalysis, the general conditions of which are familiar to those skilled in the art. The basic catalyst may be any basic catalyst known in the art, more preferably the basic catalyst is one of potassium hydroxide, sodium hydroxide, barium hydroxide and cesium hydroxide, most preferably the basic catalyst is potassium hydroxide.

Suitable initiator (or starter) compounds include, but are not limited to, C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and the like. Nominal initiator functionality is from 1 to 8 or more, preferably from 1 to 6, and more preferably from 2 to 4. The functionality of the initiators useful in the present invention may be in an amount ranging between any combination of these values, inclusive of the recited values. Any mixtures of monomeric initiators or their oxyalkylated oligomers may also be utilized.

A polyoxyethylene-containing compound, such as a polyethylene glycol, is added to chelate at least one of the cations of the basic catalyst during the alkoxylation in the inventive long-chain polyether polyol production process. Alternatively, the hydroxy functionality of the polyoxyethylene-containing compound may be capped with alkyl, preferably methyl, groups as is known to those skilled in the art. This polyoxyethylene-containing compound is added to the initiator at a level resulting in 0.5 to 20 wt. %, based on the weight of the long-chain polyether polyol, more preferably from 3 wt. % to 9 wt. %. This polyoxyethylene-containing compound preferably has a molecular weight of less than 10,000, more preferably from less than 10,000 to 100 and most preferably from 300 to 1,000 g/mole. The poloxyethylene-containing compound may have a molecular weight in an amount ranging between any combination of these values, inclusive of the recited values.

The alkylene oxides useful in alkoxylating the initiator to produce the inventive long-chain polyether polyols include, but are not limited to, ethylene oxide, propylene oxide, oxetane, 1,2- and 2,3-butylene oxide, isobutylene oxide, epichlorohydrin, cyclohexene oxide, styrene oxide, and the higher alkylene oxides such as the C₅-C₃₀ α-alkylene oxides. Propylene oxide alone or mixtures of propylene oxide with ethylene oxide or another alkylene oxide are preferred. Other polymerizable monomers may be used as well, e.g. anhydrides and other monomers as disclosed in U.S. Pat. Nos. 3,404,109, 3,538,043 and 5,145,883, the contents of which are herein incorporated in their entireties by reference thereto.

The inventive long-chain polyether polyols may preferably be reacted with a polyisocyanate, optionally in the presence of blowing agents, surfactants, cross-linking agents, extending agents, pigments, flame retardants, catalysts and fillers to produce flexible polyurethane foams or non-cellular polyurethanes.

Suitable polyisocyanates are known to those skilled in the art and include unmodified isocyanates, modified polyisocyanates, and isocyanate prepolymers. Such organic polyisocyanates include aliphatic, cycloaliphatic, araliphatic, aromatic, and heterocyclic polyisocyanates of the type described, for example, by W. Siefken in Justus Liebigs Annalen der Chemie, 562, pages 75 to 136. Examples of such isocyanates include those represented by the formula
Q(NCO)ₙ
in which n is a number from 2-5, preferably 2-3, and Q is an aliphatic hydrocarbon group; a cycloaliphatic hydrocarbon group; an araliphatic hydrocarbon group; or an aromatic hydrocarbon group.

Examples of suitable isocyanates include ethylene diisocyanate; 1,4-tetramethylene diisocyanate; 1,6-hexamethylene diisocyanate; 1,12-dodecane diisocyanate; cyclobutane-1,3-diisocyanate; cyclohexane-1,3- and -1,4-diisocyanate, and mixtures of these isomers; 1-isocyanato-3,3,5-trimethyl-5-isocyanatomethylcyclohexane (isophorone diisocyanate; German Auslegeschrift 1,202,785 and U.S. Pat. No. 3,401,190); 2,4- and 2,6-hexahydrotoluene diisocyanate and mixtures of these isomers; dicyclohexylmethane-4,4′-diisocyanate (hydrogenated MDI, or HMDI); 1,3- and 1,4-phenylene diisocyanate; 2,4- and 2,6-toluene diisocyanate and mixtures of these isomers (TDI); diphenylmethane-2,4′- and/or -4,4′-diisocyanate (MDI); polymeric diphenylmethane diisocyanate (PMDI), naphthylene-1,5-diisocyanate; triphenylmethane-4,4′,4″-triisocyanate; polyphenyl-polymethylene-polyisocyanates of the type which may be obtained by condensing aniline with formaldehyde, followed by phosgenation (crude MDI), which are described, for example, in GB 878,430 and GB 848,671; norbornane diisocyanates, such as described in U.S. Pat. No. 3,492,330; m- and p-isocyanatophenyl sulfonylisocyanates of the type described in U.S. Pat. No. 3,454,606; perchlorinated aryl polyisocyanates of the type described, for example, in U.S. Pat. No. 3,227,138; modified polyisocyanates containing carbodiimide groups of the type described in U.S. Pat. No. 3,152,162; modified polyisocyanates containing urethane groups of the type described, for example, in U.S. Pat. Nos. 3,394,164 and 3,644,457; modified polyisocyanates containing allophanate groups of the type described, for example, in GB 994,890, BE 761,616, and NL 7,102,524; modified polyisocyanates containing isocyanurate groups of the type described, for example, in U.S. Pat. No. 3,002,973, German Patentschriften 1,022,789, 1,222,067 and 1,027,394, and German Offenlegungsschriften 1,919,034 and 2,004,048; modified polyisocyanates containing urea groups of the type described in German Patentschrift 1,230,778; polyisocyanates containing biuret groups of the type described, for example, in German Patentschrift 1,101,394, U.S. Pat. Nos. 3,124,605 and 3,201,372, and in GB 889,050; polyisocyanates obtained by telomerization reactions of the type described, for example, in U.S. Pat. No. 3,654,106; polyisocyanates containing ester groups of the type described, for example, in GB 965,474 and GB 1,072,956, in U.S. Pat. No. 3,567,763, and in German Patentschrift 1,231,688; reaction products of the above-mentioned isocyanates with acetals as described in German Patentschrift 1,072,385; and polyisocyanates containing polymeric fatty acid groups of the type described in U.S. Pat. No. 3,455,883. It is also possible to use the isocyanate-containing distillation residues accumulating in the production of isocyanates on a commercial scale, optionally in solution in one or more of the polyisocyanates mentioned above. Those skilled in the art will recognize that it is also possible to use mixtures of the polyisocyanates described above. Particularly preferred in the polyurethane foams of the present invention are 2,4- and 2,6-toluene diisocyanate and mixtures of these isomers (TDI).

Prepolymers may also be employed in the preparation of the inventive foams. Prepolymers may be prepared by reacting an excess of organic polyisocyanate or mixtures thereof with a minor amount of an active hydrogen-containing compound as determined by the well-known Zerewitinoff test, as described by Kohler in Journal of the American Chemical Society, 49, 3181(1927). These compounds and their methods of preparation are known to those skilled in the art. The use of any one specific active hydrogen compound is not critical; any such compound can be employed in the practice of the present invention.

Suitable additives optionally included in the polyurethane forming formulations of the present invention include, for example, stabilizers, catalysts, cell regulators, reaction inhibitors, plasticizers, fillers, crosslinking or extending agents, blowing agents, etc.

Stabilizers which may be considered suitable for the inventive foam forming process include, for example, polyether siloxanes, and preferably those which are insoluble in water. Compounds such as these are generally of such a structure that a relatively short chain copolymer of ethylene oxide and propylene oxide is attached to a polydimethylsiloxane residue. Such stabilizers are described in, for example, U.S. Pat. Nos. 2,834,748, 2,917,480 and 3,629,308.

Catalysts suitable for the foam forming process of the present invention include those which are known in the art. These catalysts include, for example, tertiary amines, such as triethylamine, tributylamine, N-methylmorpholine, N-ethylmorpholine, N,N,N′,N′-tetramethylethylenediamine, pentamethyl-diethylenetriamine and higher homologues (as described in, for example, DE-A 2,624,527 and 2,624,528),1,4-diazabicyclo(2.2.2)octane, N-methyl-N′-dimethyl-aminoethylpiperazine, bis-(dimethylaminoalkyl)piperazines, N,N-dimethylbenzylamine, N,N-dimethylcyclohexylamine, N,N-diethyl-benzylamine, bis-(N,N-diethylaminoethyl)adipate, N,N,N′,N′-tetramethyl-1,3-butanediamine, N,N-dimethyl-β-phenylethylamine, 1,2-dimethylimidazole, 2-methylimidazole, monocyclic and bicyclic amines together with bis-(dialkylamino)alkyl ethers, such as 2,2-bis-(dimethylaminoethyl)ether.

Other suitable catalysts which may be used in producing the inventive polyurethane foams include, for example, organometallic compounds, and particularly, organotin compounds. Organotin compounds which may be considered suitable include those organotin compounds containing sulfur. Such catalysts include, for example, di-n-octyltin mercaptide. Other types of suitable organotin catalysts include, preferably tin(II) salts of carboxylic acids such as, for example, tin(II) acetate, tin(II) octoate, tin(II) ethylhexoate and/or tin(II) laurate, and tin(IV) compounds such as, for example, dibutyltin oxide, dibutyltin dichloride, dibutyltin diacetate, dibutyltin dilaurate, dibutyltin maleate and/or dioctyltin diacetate.

Water is preferably used as the sole blowing agent in the foams made according to the present invention, although auxiliary blowing agents, such as, for example, carbon dioxide, can be used. Water functions as the blowing by reacting with the isocyanate component to chemically form carbon dioxide gas plus an amine moiety which reacts further with the polyisocyanate to form urea backbone groups. Water can be used in an amount up to 10% by weight. Preferably, 1 to 8% by weight, more preferably, 1 to 5% by weight, based on the total weight of the isocyanate-reactive mixture, of water is used in the present invention.

Further examples of suitable additives, which may optionally be included in the flexible polyurethane foams of the present invention can be found in Kunststoff-Handbuch, volume VII, edited by Vieweg & Hochtlen, Carl Hanser Verlag, Munich 1993, 3^{rd} Ed., pp. 104 to 127, for example. The relevant details concerning the use and mode of action of these additives are set forth therein.

### EXAMPLES

The present invention is further illustrated, but is not to be limited, by the following examples. All quantities given in “parts” and “percents” are understood to be by weight, unless otherwise indicated. For the examples summarized below, the following materials were used:
Polyol A: a polyether polyol based on propoxylated glycerine having a hydroxyl number of 240 mg KOH/g,
Polyol B: a polyether polyol initiator based on propoxylated glycerine having a hydroxyl number of 350 mg KOH/g, contains 4 wt. % KOH;
Polyol C: a polyether polyol initiator based on propoxylated sorbitol having a hydroxyl number of 200 mg KOH/g, contains 2.2 wt. % KOH;
PEG-400: a dihydroxy terminated 400 MW polyethylene glycol (Aldrich Chemical Co.);
PEG-1000: a dihydroxy terminated 1000 MW polyethylene glycol (Aldrich Chemical Co.);
PEG-10000: a dihydroxy terminated 10,000 MW polyethylene glycol (Aldrich Chemical Co.);
PEG-100000: a dihydroxy terminated 100,000 MW polyethylene glycol (Aldrich Chemical Co.);
PEG-500 dimethylether: a dimethoxy terminated 500 MW polyethylene glycol (Aldrich Chemical Co.); and
PEG-1000 dimethylether: a dimethoxy terminated 1000 MW polyethylene glycol (Aldrich Chemical Co.).

### Example C-1

In this comparative example, Polyol A (190 g) and 50% aqueous KOH (4.74 g) were charged to a one-liter polyether polyol reactor. The mixture was stripped for 30 minutes under vacuum (˜0.5 psia) with a nitrogen purge at 110° C. to remove water. The nitrogen purge was stopped and vacuum valve to the reactor was closed, thus blocking the vacuum (0.5 psia) in the reactor. Propylene oxide (300 g) was fed to the reactor using a pressure feed back loop to control feed rate to maintain 50 psia pressure in the reactor throughout the process. The time required to add the propylene oxide was recorded and used to determine absolute feed rate (g/min).

### Examples 2-4

Polyol A (see Table I for charge weight), 50% aqueous KOH (4.68 g) and PEG-400 (see Table I for charge weight) were charged to a one-liter polyether polyol reactor. The mixture was stripped for 30 minutes under vacuum (˜0.5 psia) with a nitrogen purge at 110° C. to remove water. The nitrogen purge was stopped and vacuum valve to the reactor was closed, thus blocking the vacuum (0.5 psia) in the reactor. Propylene oxide (300 g) was fed to the reactor using a pressure feed back loop to control feed rate to maintain 50 psia pressure in the reactor throughout the run. The time required to add the propylene oxide was recorded and used to determine absolute feed rate (g/min).

The feed rate for the examples prepared with a polyoxyethylene containing additive according to the invention (Ex. 2-4) are shown along with comparison Example C-1 (prepared without a polyoxythyelene-containing additive) in Table I. As can be appreciated by reference to Table I, it was found that the rate of the KOH-catalyzed propoxylation reaction at 110° C. could be accelerated by approximately 45-50% with incorporation of about 9 wt. % of PEG-400-and approximately 15-20% with about 3 wt. % PEG-400.

TABLE I

| | C-1 | Ex. 2 | Ex. 3 | Ex. 4 |
|---|---|---|---|---|
| Polyol A | 190 | 169 | 140 | 140 |
| PEG-400 (g) | — | 14 | 42.8 | 42.8 |
| Propylene Oxide (g) | 300 | 300 | 300 | 300 |
| PEG-400 (wt. %) | — | 2.9 | 8.8 | 8.8 |
| PO Addition time (min.) | 211 | 180 | 140 | 143 |
| PO Addition Rate (g/min) | 1.43 | 1.67 | 2.14 | 2.10 |
| Relative Rate PO Addition | 1.0 | 1.17 | 1.50 | 1.47 |

Based on these results, the inventive concept was extended to a start mixture representative of that used to prepare polyether polyols for molded foam applications. The effectiveness of PEG additives having different molecular weights and either-hydroxyl or methoxy end groups were evaluated, and the results are presented in Table II below.

### Example C-5

In this comparative example, a start mixture having a hydroxyl number of 290 mg KOH/g was prepared from 60% Polyol B (120 g) and 40% Polyol C (80 g). This mixture was charged to a one-liter stainless steel polyether polyol reactor. The start mixture was heated under vacuum (˜0.5 psia) at 105° C., while allowing nitrogen to flow through the reactor. After 30 minutes, the nitrogen feed was stopped, and the vacuum valve was closed, thus blocking the vacuum in the reactor (˜0.5 psia). Propylene oxide (400 g) was fed into the reactor at a rate sufficient to maintain 40 psia reactor pressure. The time required to complete the PO feed was measured and used to calculate a feed rate (g/min.) for the standard propoxylation.

### Examples 6-10 and C-11

A start mixture similar to that of Example C-5 was prepared, except a portion of Polyol B was replaced gram for gram with the indicated polyoxyethylene containing compound (see Table II). Sufficient KOH was added as a 50% aqueous mixture (3.76 g) to bring the total KOH level to the same as that of Example C-5. This mixture was charged to a one-liter stainless steel polyether polyol reactor. The start mixture was heated under vacuum (˜0.5 psia) at 105° C., while allowing nitrogen to flow through the reactor. After 30 minutes, the nitrogen feed was stopped, and the vacuum valve was closed, thus blocking the vacuum in the reactor. Propylene oxide (400 g) was fed into the reactor at a rate sufficient to maintain 40 psia reactor pressure. The time required to complete the 400 g feed was measured and used to calculate a feed rate (g/min.)

The data for Example C-5 (no polyoxythyelene-containing additive) and Examples 6-10 and C-11, with various polyoxyethylene containing compounds having different MW and end groups, are summarized in Table II below.

TABLE II

| | C-5 | Ex. 6 | Ex. 7 | Ex. 8 | Ex. 9 | Ex. 10 | C- 11 |
|---|---|---|---|---|---|---|---|
| Polyol B | 120 | 73 | 73 | 73 | 73 | 73 | 73 |
| Polyol C | 80 | 80 | 80 | 80 | 80 | 80 | 80 |
| PEG-400 (g) | | 47 | | | | | |
| PEG-500 dimethylether | | | 47 | | | | |
| PEG-1000 (g) | | | | 47 | | | |
| PEG 1000 dimethylether | | | | | 47 | | |
| PEG 10,000 | | | | | | 47 | |
| PEG 100,000 | | | | | | | 47 |
| Propylene Oxide (g) | 400 | 400 | 400 | 400 | 400 | 400 | 400 |
| PEG (wt. %) | — | 7.8 | 7.8 | 7.8 | 7.8 | 7.8 | 7.8 |
| PO Addition time (min.) | 167 | 125 | 136 | 131 | 132 | 143 | 195 |
| PO Addition Rate (g/min) | 2.39 | 3.20 | 2.94 | 3.05 | 3.03 | 2.80 | 2.05 |
| Relative Rate PO Addition | 1.00 | 1.34 | 1.23 | 1.28 | 1.27 | 1.17 | 0.86 |
| Product Appearance | Clear | Clear | Clear | Clear | Clear | Contains | Contains |
| | liquid | liquid | liquid | liquid | liquid | solids | solids |

As can be appreciated by reference to Table II, the polyoxyethylene containing additives having molecular weight ranging from 400-10,000 g/mole led to increased rate of KOH-catalyzed propoxylation, regardless of the end-group type (hydroxyl or methoxy). Interestingly, at the same level as the other PEGs, the PEG-100,000 (Ex. C-11) additive decreased the rate of propoxylation.

Although not wishing to be limited to any particular theory, the inventors herein speculate that the high molecular weight PEG resides in a separate phase and carries with it some of the KOH catalyst resulting in overall slower propoxylation rate. Examining the products after the final propoxylation showed that the PEG-10,000 (Ex. 10) and PEG-100,000 (Ex. C-11) containing products contained solids. The 1,000 and lower molecular weight PEGs, at the levels investigated, gave liquid products without evidence for any solids. Liquid polyether polyols that do not contain solids are generally easier to process into polyurethanes and are generally recognized as higher quality. This, dependence of the polyether quality on the molecular weight of the PEG additive, together with decreased effectiveness of the higher molecular weight PEGs in accelerating the propoxylation has, to the best of the inventors' knowledge, heretofore not been taught in the art.

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

1. A long-chain polyether polyol having a number average molecular weight of more than about 1,200 g/mole and produced by alkoxylating an initiator with an alkylene oxide in the presence of a basic catalyst having at least one cation thereof chelated with about 0.5 to about 20 wt. % of a polyoxyethylene-containing compound having a molecular weight of less than about 10,000 g/mole, wherein the weight percentage is based on the weight of the long-chain polyether polyol.

2. The long-chain polyether polyol according to claim 1 having a number average molecular weight of from about 1,200 g/mole to about 50,000 g/mole.

3. The long-chain polyether polyol according to claim 1 having a number average molecular weight of from about 1,200 g/mole to about 30,000 g/mole.

4. The long-chain polyether polyol according to claim 1 having a number average molecular weight of from about 1,200 g/mole to about 8,000 g/mole.

5. The long-chain polyether polyol according to claim 1, wherein the initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

6. The long-chain polyether polyol according to claim 1, wherein the basic catalyst is chosen from potassium hydroxide, sodium hydroxide, barium hydroxide and cesium hydroxide.

7. The long-chain polyether polyol according to claim 1, wherein the basic catalyst is potassium hydroxide.

8. The long-chain polyether polyol according to claim 1, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

9. The long-chain polyether polyol according to claim 1, wherein the alkylene oxide is propylene oxide or a block of propylene oxide followed by a blocktof ethylene oxide.

10. The long-chain polyether polyol according to claim 1, wherein the polyoxyethylene-containing compound has a molecular weight of from less than about 10,000 g/mole to about 100 g/mole.

11. The long-chain polyether polyol according to claim 1, wherein the polyoxyethylene-containing compound has a molecular weight of from about 300 g/mole to about 1000 g/mole.

12. The long-chain polyether polyol according to claim 1, wherein the at least one cation of the basic catalyst is chelated with about 3 wt. % to about 9 wt. % of the polyoxyethylene-containing compound.

13. A process for producing a long chain polyether polyol having a number average molecular weight of at least about 1,200 g/mole, the process comprising:
alkoxylating an initiator with an alkylene oxide in the presence of a basic catalyst having at least one cation thereof chelated with about 0.5 to about 20 wt. % of a polyoxyethylene-containing compound having a molecular weight of less than about 10,000 g/mole,
wherein the weight percentage is based on the weight of the long-chain polyether polyol.

14. The process according to claim 13, wherein the long chain polyether polyol has a number average molecular weight of from about 1,200 g/mole to about 50,000 g/mole.

15. The process according to claim 13, wherein the long chain polyether polyol has a number average molecular weight of from about 1,200 g/mole to about 30,000 g/mole.

16. The process according to claim 13, wherein the long chain polyether polyol has a number average molecular weight of from about 1,200 g/mole to about 8,000 g/mole.

17. The process according to claim 13, wherein the initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

18. The process according to claim 13, wherein the basic catalyst is chosen from potassium hydroxide, sodium hydroxide, barium hydroxide and cesium hydroxide.

19. The process according to claim 13, wherein the basic catalyst is potassium hydroxide.

20. The process according to claim 13, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

21. The process according to claim 13, wherein the alkylene oxide is propylene oxide or a block of propylene oxide followed by a block of ethylene oxide.

22. The process according to claim 13, wherein the polyoxyethylene-containing compound has a molecular weight of from less than about 10,000 g/mole to about 100 g/mole.

23. The process according to claim 13, wherein the polyoxyethylene-containing compound has a molecular weight of from about 300 g/mole to about 1,000 g/mole.

24. The process according to claim 13, wherein the at least one cation of the basic catalyst is chelated with about 3 wt. % to about 9 wt. % of the polyoxyethylene-containing compound.

25. A flexible polyurethane foam comprising the reaction product of at least one polyisocyanate; and
at least one long-chain polyether polyol having a number average molecular weight of more than about 1,200 g/mole and produced by alkoxylating an initiator with an alkylene oxide in the presence of a basic catalyst having at least one cation thereof chelated with about 0.5 to about 20 wt. % of a polyoxyethylene-containing compound having a molecular weight of less than about 10,000 g/mole,
optionally in the presence of at least one of blowing agents, surfactants, cross-linking agents, extending agents, pigments, flame retardants, catalysts and fillers
wherein the weight percentage is based on the weight of the long-chain polyether polyol.

26. The flexible polyurethane foam according to claim 25, wherein the at least one polyisocyanate is chosen from ethylene diisocyanate, 1,4-tetra-methylene diisocyanate, 1,6-hexamethylene diisocyanate, 1,12-dodecane diisocyanate, cyclobutane-1,3-diisocyanate, cyclohexane-1,3-and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diiso-cyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-terminated prepolymers and mixtures thereof.

27. The flexible polyurethane foam according to claim 25, wherein the at least one polyisocyanate is chosen from 2,4- and 2,6-toluene diisocyanate and mixtures thereof (TDI).

28. The flexible polyurethane foam according to claim 25, wherein the initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butane-diol, 1,6-hexanediol, glycerin, trimethylolpropane, trimethylolethane, pentaerythritol, α-methylglucoside, sorbitol, mannitol, hydroxymethylglucoside, hydroxypropyl-glucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

29. The flexible polyurethane foam according to claim 25, wherein the basic catalyst is chosen from potassium hydroxide, sodium hydroxide, barium hydroxide and cesium hydroxide.

30. The flexible polyurethane foam according to claim 25, wherein the basic catalyst is potassium hydroxide.

31. The flexible polyurethane foam according to claim 25, wherein the polyoxyethylene-containing compound has a molecular weight of from less than about 10,000 g/mole to about 100 g/mole.

32. The flexible polyurethane foam according to claim 25, wherein the polyoxyethylene-containing compound has a molecular weight of from about 300 g/mole to about 1,000 g/mole.

33. The flexible polyurethane foam according to claim 25, wherein the long-chain polyether polyol has a number average molecular weight of from about 1,200 g/mole to about 50,000 g/mole.

34. The flexible polyurethane foam according to claim 25, wherein the long-chain polyether polyol has a number average molecular weight of from about 1,200 g/mole to about 30,000 g/mole.

35. The flexible polyurethane foam according to claim 25, wherein the long-chain polyether polyol has a number average molecular weight of from about 1,200 g/mole to about 8,000 g/mole.

36. The flexible polyurethane foam according to claim 25, wherein the at least one cation of the basic catalyst is chelated with about 3 wt. % to about 9 wt. % of the polyoxyethylene-containing compound.

37. A process for producing a flexible polyurethane foam comprising reacting
at least one polyisocyanate; and
at least one long-chain polyether polyol having a number average molecular weight of more than about 1,200 g/mole and produced by alkoxylating an initiator with an alkylene oxide in the presence of a basic catalyst having at least one cation thereof chelated with about 0.5 to about 20 wt. % of a polyoxyethylene-containing compound having a molecular weight of less than about 10,000 g/mole,
optionally in the presence of at least one of blowing agents, surfactants, cross-linking agents, extending agents, pigments, flame retardants, catalysts and fillers
wherein the weight percentage is based on the weight of the long-chain polyether polyol.

38. The process according to claim 37, wherein the at least one polyisocyanate is chosen from ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3-and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diisocyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-terminated prepolymers and mixtures thereof.

39. The process according to claim 37, wherein the at least one polyisocyanate is chosen from 2,4- and 2,6-toluene diisocyanate and mixtures thereof (TDI).

40. The process according to claim 37, wherein the initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

41. The process according to claim 37, wherein the basic catalyst is chosen from potassium hydroxide, sodium hydroxide, barium hydroxide and cesium hydroxide.

42. The process according to claim 37, wherein the basic catalyst is potassium hydroxide.

43. The process according to claim 37, wherein the long-chain polyether polyol has a number average molecular weight of from about 1,200 g/mole to about 50,000 g/mole.

44. The process according to claim 37, wherein the long-chain polyether polyol has a number average molecular weight of from about 1,200 g/mole to about 30,000 g/mole.

45. The process according to claim 37, wherein the long-chain polyether polyol has a number average molecular weight of from about 1,200 g/mole to about 8,000 g/mole.

46. The process according to claim 37, wherein the polyoxyethylene-containing compound has a molecular weight of from less than about 10,000 g/mole to about 100 g/mole.

47. The process according to claim 37, wherein the polyoxyethylene-containing compound has a molecular weight of from about 300 g/mole to about 1,000 g/mole.

48. The process according to claim 37, wherein the at least one cation of the basic catalyst is chelated with about 3 wt. % to about 9 wt. % of the polyoxyethylene-containing compound.
